(19) United States
(12) Patent Application Publication
(10) Pub. No.: US 2006/0093653 A1
(43) Pub. Date: May 4, 2006

(54) Periadventitial delivery device

(76) Inventors: John Francis Martin, London (GB); Seppo Yla-Herttuala, Kuopio (FI); Stephen George Edward Barker, London (GB)

Correspondence Address: SALIWANCHIK LLOYD & SALIWANCHIK;A PROFESSIONAL ASSOCIATION, PO BOX 142950, GAINESVILLE, FL, 32614-2950, US

(21) Appl. No.: 11/253,285

(22) Filed: Oct. 18, 2005

Related U.S. Application Data
(63) Continuation of application No. 10/196,345, filed on Jul. 15, 2002, now abandoned.
Application No. 10/196,345 is a continuation of application No. 09/674,232, filed on Oct. 27, 2000, now abandoned.

(30) Foreign Application Priority Data
Apr. 28, 1998 (GB) 9809082.2
May 14, 1998 (GB) 9810429.2

(51) Int. Cl. A61K 48/00; A61K 9/70

(52) U.S. Cl. 424/443; 514/44

(57) ABSTRACT

The subject invention pertains to a biodegradable matrix material which is provided in a form that can be wrapped around a body part, in combination or impregnated with an agent that can be delivered to treat a condition via the adventitial surface of a body part, the agent being in a form that can be taken up by the matrix material. These components are provided for use in the treatment of the condition, for example, by using a sealant to form a seal around the matrix material when impregnated with the agent.

### FIELD OF THE INVENTION

The present invention relates to a device that can be used for delivering an active agent, in therapy.

### BACKGROUND OF THE INVENTION

Intimal hyperplasia is the increase in the number of cells between the endothelium and internal elastic lamina of a blood vessel, particularly in the intimal layer found there, or in an artery. Intimal hyperplasia is often caused by smooth muscle cell (SMC) proliferation in the blood vessel wall.

When intimal hyperplasia occurs, de novo thickening of the intimal layer or of the vessel wall, i.e. stenosis, may result. Thus, the blood vessel may become occluded.

Also, when an obstruction in a blood vessel has been cleared, intimal hyperplasia occurring after surgery may lead to the artery's becoming occluded again. This is known as restenosis.

Intimal hyperplasia, whether it leads to stenosis or restenosis, remains a major problem after various surgical procedures.

GB-A-2298577 discloses a non-restrictive, porous, external stent for arteriovenous bypass grafting procedures. This stent has beneficial effects on luminal size and on medial and intimal thickening.

WO-A-9423668 discloses a device for the local delivery of an agent into a blood vessel, including a reservoir formed between two elements thereof. Its use requires implantation, i.e. cutting through the vessel and then securing the device to the vessel walls. The device is partially porous. The reservoir is in direct contact with luminal blood flow. This involves the risk of infection.

U.S. Pat. No. 3,797,485 discloses a device for delivering a drug to the adventitial surface of a blood vessel. It is provided with permanent walls and transcutaneous tubes for the delivery of drug in liquid form. The intention is that the drug should pass to another site.

U.S. Pat. No. 5,540,928 and related patent publications (inventors: Edelman et al) disclose an extraluminal device in the form of a disc comprising a polymer matrix, with a central hole. In order to ensure that the agent, e.g. heparin, is delivered at the blood vessel wall, a radial hole may be bored in the coating; see Edelman et al, PNAS USA 87:3773-7 (May 1990).

### SUMMARY OF THE INVENTION

The present invention is based on initial experiments in which a collar was placed around the outside of the artery of a rabbit. This procedure normally causes intimal hyperplasia in the rabbit artery, leading to thickening of the arterial wall, which is similar to the stenosis that can occur in human arteries following bypass operations. When the collar was used to deliver DNA encoding VEGF to the arterial wall using a plasmid/liposome vector, the VEGF gene was overexpressed in the arterial wall, including the endothelial layer. Intimal hyperplasia was inhibited. It has been found that adventitial delivery is suitable for all tested systems.

The new findings demonstrate that effective agents can be delivered to the exterior of the blood vessel, to treat intimal hyperplasia. This has several advantages. In particular, the therapeutic agent is not washed away from the site of the hyperplasia by blood flow as with intralumenal delivery. A delivery reservoir can be maintained around the blood vessel, and there is no need for any intralumenal manipulations which damage the endothelium of the blood vessel (and can themselves trigger intimal hyperplasia).

According to one aspect of the invention, a device for use in the delivery of a therapeutic agent to a blood vessel or other elongate, internal member in a patient, comprises an outer layer adapted to provide a seal around the member, the agent being held within or associated with the device so that, in use, the agent comes into contact with the outer surface of the member. Such devices can be biodegradable, and do not require permanent transcutaneous delivery tubes.

One aspect of a method of application of a therapeutic agent, according to the invention, comprises surgical exposure of a body part; application, around the part, of an outer layer or of a matrix material; introduction of a pharmaceutical formulation containing the agent, either into the volume defined between the outer layer and the outer surface of the body part or into the matrix material (followed by providing a seal around the matrix material); and closure of the surgical wound.

### DESCRIPTION OF THE INVENTION

Various agents, including peptidic and non-peptidic compounds, genes that can express active products etc, are suitable for use in the invention. As described in WO-A-9820027 (the content of which is incorporated herein by reference), an illustrative agent is the VEGF protein or nucleic acid. Herein, references to such agents, and to VEGF itself, are given by way of example.

Nucleic acids may be delivered in a “naked” form unassociated with a vector, or by means of a gene therapy vector. It is preferred to deliver them by means of any suitable gene therapy vector. In particular, viral or non-viral vectors may be used.

The body part to which the invention may be applied is typically a duct, and will typically be essentially tubular or cylindrical. For example, it may be a nerve, Fallopian tube, bile duct, aortic aneurism or blood vessel. In particular, anti-thrombotic agents may be administered to act on blood platelets or the coagulation cascade, growth factors to the nerves, and anti-rejection agents to transplanted organs.

For example, the active agent may be delivered to the outside of the body part to be treated, e.g. artery. This may be achieved by means of an implant placed externally to the blood vessel, in proximity to a site of hyperplasia to be treated. Such an implant may contain VEGF protein or nucleic acid or the vector and provides a reservoir of the agent.

The agent (preferably in association with a vector) may be introduced into the implant before or after the implant is introduced into the subject to be treated. For example, the implant may be fitted in the vicinity of the blood vessel; the agent is introduced into the implant, e.g. by injection, subsequently.

Preferably, the implant is placed in direct contact with the blood vessel, e.g. artery. This is especially preferred when retroviral vectors are used to deliver nucleic acids, as the physical distortion of the blood vessel may induce smooth muscle cell proliferation, which increases the efficiency of gene transfer by retroviral vectors. This proliferation, like the proliferation induced by the hyperplasia itself, is overcome or at least ameliorated, by the delivery of the agent. Similarly, it is preferred for the implant to be in contact with the artery when employing other vectors that exhibit increased efficiency of gene transfer when their target cells are dividing. For example, cell proliferation may also enhance gene transfer efficiency with plasmid/liposome complexes.

Such implants may be in any suitable form. An implant in the form of a collar which surrounds, partially or completely, preferably completely, the artery, at or near the site of the hyperplasia to be treated or prevented, is fully described and illustrated (see the drawings) in WO-A-9820027.

Extravascular delivery avoids procedures such as balloon catheterization or high pressure fluid which may lead to endothelial damage or denudation. Transfected genes are preferably applied via a silastic or biodegradable implant, placed next to, preferably around, the outside of the blood vessel. The endothelium suffers little or no damage. This is a major advantage of this form of delivery.

Implants may be made of any suitable material. Silastic implants, i.e. implants comprising silicone rubbers, are one preferred alternative. Most preferred are biodegradable implants. Any suitable biodegradable material may be used.

In a preferred aspect of this invention, treatment comprises surgical exposure of the body part; application, around the part, of a strip of a matrix material including, or to include, the agent; covering the matrix material with an outer, sealing layer; and closure of the surgical wound.

Particularly in this latter aspect, the agent may be contained within a medium within the device, e.g. a solid or gel medium. This may help to prevent the agent escaping into the tissue.

For example, a sheet or strip of a biodegradable material may be impregnated with the therapeutic agent. The strip is cut to a desired size, before or after being wound around the body part to be treated. It is then sealed in situ by the application of, for example, a tissue glue around the matrix material. The glue may advantageously be activated remotely, e.g. by light.

Alternatively, the agent may be coated onto the surface of the implant which is in contact with the body part, in use. Alternatively, the agent may be dispersed throughout the structure of the implant.

Some advantages of such implants are that: (i) they provide a delivery reservoir, allowing for sustained delivery; (ii) no intralumenal manipulations are required and the, say, arterial endothelium remains intact; and (iii) the distortion (e.g. constriction in the case of a collar) created by the implant may enhance the efficiency of gene delivery, as explained above.

The invention provides a relatively or substantially impermeable outer layer. It may provide a diffusion barrier.

As indicated above, the therapeutic agent that is used in the present invention may be a nucleic acid from which a gene product is derived, in situ, e.g. following transport across the wall of the body part to which the device is applied. By way of example, a suitable gene may be provided in a polymer solution. If it is desired that a long-acting effect is provided, continuous expression of a gene may be provided, e.g. using fibroblasts.

The present invention may be understood with reference to the accompanying drawing, in which:

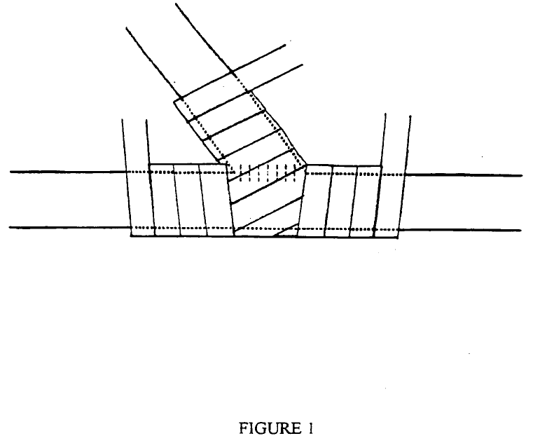

FIG. 1 is a schematic view of a “wrap” embodying the invention in place around an arterial anastomosis.

Particularly where a strip of flexible matrix material is used as a wrap, it may be provided in a kit with a sealant and the agent. These components may be separate, or two or more may be combined. Thus, the agent may be pre-impregnated in the matrix material. The material may be in bi-layer form, one layer being of the matrix and the other of a relatively impermeable material, e.g. both of collagen but of different characteristics. Any or each component may be aseptically packaged, in generally known manner, ready for use.

The sealant may be a conventional “tissue glue”, such as the thrombin glue sold under the name Tisseal, or a cyanomethacrylate-based glue.

The matrix material is, advantageously, biodegradable over a set time course, for example a period of 1 to 5 days, by which time the active agents in the formulation are likely to have become exhausted. The material is also chosen so as not to promote too severe a reaction from the surrounding tissue. Examples of suitable materials for the body include gelatin, alginate or collagen. These materials also allow the body flexibility and enable the device to be manufactured by molding or extrusion.

The outer layer may, for example, be made of solid collagen and the inner layer made of sponge-like collagen cross-linked thereto, the sponge-like layer being capable of being impregnated with the pharmaceutical formulation containing the agent to be delivered. In such a situation, the device may be provided to the surgeon for fitment with the formulation already impregnated therein, or it may be wetted with the formulation after fitment, for example by being injected as described earlier.

Alternatively, the agent may be coated onto an internal surface of the body, which surface is just in contact with the blood vessel in use. Alternatively, agent may be dispersed throughout the structure of the body.

It is desirable that the body of the device should have sufficient strength to resist torsional forces. For this purpose, the body may be formed with, for example, an inner layer, e.g. a collagen film, or longitudinal, transverse or helical ribs. Ribs may be provided that subdivide the reservoir into compartments, and to provide additional stability.

As indicated above, VEGF proteins or nucleic acids may be used for the treatment or prevention of intimal hyperplasia arising from any clinical circumstances. For example, it is possible to treat hyperplasia arising after any type of surgical procedure, including angioplasty, for example balloon angioplasty; bypass surgery, such as coronary bypass surgery in which a vein is anastomosed to an artery; other anastomosis procedures, for example anastomosis in the legs; and endarteriectomy, for example carotid artery endarteriectomy. It is also possible to treat intimal hyperplasia associated with arterial damage or hypertension, for example pulmonary artery hypertension. The invention provides for treatment of intimal hyperplasia in any type of blood vessel, e.g. in an artery or vein, preferably an artery.

According to the invention, it is possible to treat or ameliorate established intimal hyperplasia or to prevent intimal hyperplasia from arising. Similarly, it is possible to diminish the likelihood of intimal hyperplasia arising, or to diminish the severity of established intimal hyperlasia or hyperplasia that is likely to arise. Treatment according to the invention may take place before, during, or after a surgical procedure, for example in order to reduce the chance of hyperplasia arising after the procedure.

Preferably, the VEGF nucleic acid or protein is administered with a view to preventing or treating de novo stenosis. It can, however, also be used to treat or prevent restenosis.

The proteins or nucleic acids of the invention are preferably delivered in the form of a pharmaceutical formulation comprising a pharmaceutically acceptable carrier. Any suitable pharmaceutical formulation may be used.

For example, suitable formulations may include aqueous and non-aqueous sterile injection solutions which may contain anti-oxidants, buffers, bacteriostats, bactericidal antibiotics and solutes which render the formulation isotonic with the blood of the intended recipient; and aqueous and non-aqueous sterile suspensions which may include suspending agents and thickening agents. The formulations may be presented in unit-dose or multi-dose containers, for example sealed ampoules and vials, and may be stored in a frozen or freeze-dried (lyophilized) condition requiring only the addition of the sterile liquid carrier, for example water for injection, immediately prior to use.

It should be understood that, in addition to the ingredients particularly mentioned above, formulations of this invention may include other agents conventional in the art having regard to the type of formulation in question. Of the possible formulations, sterile pyrogen-free aqueous and non-aqueous solutions are preferred.

The proteins, nucleic acids and vectors may be delivered in any suitable dosage, and using any suitable dosage regime. Those of skill in the art will appreciate that the dosage amount and regime may be adapted to ensure optimal treatment of the particular condition to be treated, depending on numerous factors. Some such factors may be the age, sex and clinical condition of the subject to be treated.

For the delivery of naked nucleic acids encoding VEGF or constructs comprising such nucleic acids, typical doses are from 0.1-5000 μg, for example 50-2000 μg, such as 50-100 μg, 100-500 μg or 500-2000 μg per dose. For the delivery of VEGF protein, suitable doses include doses of from 1 to 1000 μg for example from 1 to 10 μg, from 10 to 100 μg, from 100 to 500 μg or from 500 to 1000 μg.

One embodiment of the invention involves the perivascular delivery of liposomally-associated human VEGF₁₆₅ gene to the popliteal artery of patients with severe peripheral vascular disease undergoing above-knee amputation. This may comprise placing a perivascular gene delivery system in the form of a wrap in position around the popliteal artery and sealing with tissue glue.

The agents to be administered, e.g. an aqueous solution of gene polasmid/liposome complexes, is delivered locally to the target tissue by soaking a strip of collagen sheet with the solution immediately before it is applied to the popliteal artery.

The collagen wrap is a strip cut from surgical collagen sheet of 25 mm long and 4-5 mm wide. It is saturated with 2.0 ml of solution of the agent, containing the dose of gene plasmid, and then wrapped around a 25 mm long segment of the popliteal artery. It is then covered completely with two layers of surgical sealant.

1. A product comprising:
a biodegradable matrix material in a form that can be wrapped around a branching blood vessel;
a cell-free nucleic acid encoding a VEGF protein, to treat a condition via delivery to the adventitial surface of the blood vessel, the cell-free nucleic acid being in a form that can be taken up by the matrix material; and
a sealant that is external to the matrix material;
for combined use in the treatment of the condition, by using the sealant to form a seal around the matrix material when the matrix material is impregnated with the cell-free nucleic acid.

2. The product according to claim 1, wherein the matrix material is impregnated with the cell-free nucleic acid.

3. The product according to claim 1, wherein the matrix material and the cell-free nucleic acid are separate.

4. The product according to claim 1, wherein the sealant is a glue.

5. The product according to claim 1, wherein the form of the matrix material is a flexible strip.

6. The product according to claim 1, wherein the matrix material comprises collagen.

7. The product according to claim 1, wherein the condition to be treated is stenosis or restenosis of the blood vessel.

8. A product to treat a condition via delivery of a cell-free nucleic acid encoding a VEGF protein to the adventitial surface of a blood vessel, comprising, aseptically packaged, a biodegradable matrix material, in a form that can be wrapped around a branching blood vessel, wherein the matrix material is impregnated with the cell-free nucleic acid.

9. The product according to claim 8, wherein the form of the matrix material is a flexible strip.

10. The product according to claim 8, wherein the matrix material comprises collagen.

11. The product according to claim 8, wherein the condition to be treated is stenosis or restenosis of the blood vessel.

12. A method for treating or inhibiting one or more conditions selected from stenosis, restenosis or intimal hyperplasia, comprising applying a cell-free nucleic acid encoding a VEGF protein to a blood vessel of a patient's body, which comprises surgical incision of epithelial tissue to expose the blood vessel; application of an outer layer that can be wrapped around the blood vessel; introduction of a pharmaceutical formulation containing the cell-free nucleic acid into the volume defined between the outer layer and the outer surface of the blood vessel, wherein the endothelium of the blood vessel suffers little or no damage; and closure of the surgical wound, including the epithelium of the patient's body, whereby the condition is inhibited or reduced at the site of application of the cell-free nucleic acid.

13. The method according to claim 12, for the treatment or inhibition of stenosis or restenosis of the blood vessel.

14. A method for treating or inhibiting one or more conditions selected from stenosis, restenosis or intimal hyperplasia, applying a cell-free nucleic acid encoding a VEGF protein to a blood vessel of a patient's body, which comprises surgical incision of epithelial tissue to expose the blood vessel, application of a strip of a matrix material that can be wrapped around the blood vessel and including the cell-free nucleic acid around the blood vessel; covering the matrix material with an outer, sealing layer, wherein the endothelium of the blood vessel suffers little or no damage; and closure of the surgical wound, including the epithelium of the patient's body, whereby the condition is inhibited or reduced at the site of application of the cell-free nucleic acid.

15. The method according to claim 14, wherein the matrix material is impregnated with the cell-free nucleic acid.

16. The method according to claim 14, wherein the matrix material and the cell-free nucleic acid are separate.

17. The method according to claim 14, wherein the outer sealing layer is a glue.

18. The method according to claim 14, wherein the form of the matrix material is a flexible strip.

19. The method according to claim 14, wherein the matrix material comprises collagen.

20. The method according to claim 14, for the treatment or inhibition of stenosis or restenosis of the blood vessel.

21. The product according to claim 1, wherein the VEGF protein is a human VEGF protein.

22. The product according to claim 8, wherein the VEGF protein is a human VEGF protein.

23. The method according to claim 12, wherein the VEGF protein is a human VEGF protein.

24. The method according to claim 14, wherein the VEGF protein is a human VEGF protein.

25. The method according to claim 12, wherein the outer layer is wrapped around a branching blood vessel.

26. The method according to claim 14, wherein the matrix material is wrapped around a branching blood vessel.
